(19) United States
(12) Patent Application Publication
(10) Pub. No.: US 2024/0351010 A1
(43) Pub. Date: Oct. 24, 2024

(54) METHODS OF CONVERTING TOLUENE TO BENZENE AND XYLENE USING MESOPOROUS MORDENITE

(71) Applicant: Saudi Arabian Oil Company, Dhahran (SA)

(72) Inventors: Veera Venkata Ramakrishna Tammana, Dhahran (SA); Ali S. Abdali, Al-Khobar (SA)

(73) Assignee: Saudi Arabian Oil Company, Dhahran (SA)

(21) Appl. No.: 18/623,574

(22) Filed: Apr. 1, 2024

Related U.S. Application Data
(60) Provisional application No. 63/497,849, filed on Apr. 24, 2023.

(51) Int. Cl. B01J 29/04; B01J 29/26; B01J 35/30; C07C 6/12; C07C 15/04; C07C 15/08

(52) U.S. Cl. CPC B01J 29/045; B01J 29/26; B01J 35/391; C07C 6/123; C07C 15/04; C07C 15/08; B01J 2229/18; B01J 2229/38; C07C 2529/26

(57) ABSTRACT

Described herein are methods for converting toluene to benzene and xylene. The methods include contacting a stream comprising toluene with a hierarchical zeolite catalyst in the presence of hydrogen gas to produce the benzene and xylene via toluene disproportionation. The hierarchical zeolite catalyst includes mesoporous Mordenite. The mesoporous Mordenite includes a plurality of mesopores and at least a portion of the plurality of mesopores are uniformly arranged in an MCM-41 framework.

### CROSS-REFERENCE TO RELATED APPLICATIONS

This application claims the benefit of U.S. Provisional Application Ser. No. 63/497,849 filed Apr. 24, 2023, the contents of which are incorporated in their entirety herein.

### FIELD

The embodiments described herein generally relate to uses of porous materials and, more particularly, uses of zeolites.

### BACKGROUND

Aromatic hydrocarbon compounds derived from petrochemical sources, benzene (C₆H₆), toluene (methylbenzene, C₇H₈), and xylenes (dimethylbenzenes, C₈H₁₀ isomers) may be used as starting materials for a wide range of consumer products. The xylenes include three isomers of dimethylbenzene, namely: 1,2-dimethylbenzene (ortho-xylene or o-xylene), 1,3-dimethylbenzene (meta-xylene or m-xylene), and 1,4-dimethylbenzene (para-xylene or p-xylene). The three isomers of xylene may be used in the synthesis of a number of useful products. For instance, upon oxidation, the p-xylene isomer yields terephthalic acid, which may be used in the manufacture of polyester plastics and synthetic textile fibers (such as Dacron®), films (such as Mylar®), and resins (such as polyethylene terephthalate, used in making plastic bottles). The m-xylene isomer may be used in the manufacture of plasticizers, azo dyes, and wood preservers. The o-xylene isomer may be used as a feedstock for phthalic anhydride production, which in turn may be used to make polyesters, alkyl resins, and PVC plasticizers. Therefore, the demand for xylenes remains strong as markets for polyester fibers and polyethylene terephthalate continue to demonstrate high growth rates.

A major source of xylenes is catalytic reformate, which is produced by contacting petroleum naphtha with a hydrogenation/dehydrogenation catalyst on a support. The resulting reformate is a complex mixture of paraffins and C₆ to C₈ aromatics, in addition to a significant quantity of heavier aromatic hydrocarbons. After removing the light (C₅ or less) paraffinic components, the remainder of reformate is normally separated over a number of distillation steps into fractions containing C₆, C₇, C₈, and C₉₊ hydrocarbons. Toluene is recovered during this process. Although toluene is an important aromatic hydrocarbon, as previously discussed, the demand for benzene and xylenes, particularly para-xylene, is much higher. Thus, toluene is generally used to produce these other aromatics.

### SUMMARY

Transalkylation reactions for converting aromatic hydrocarbon compounds to compounds having a different number of carbon atoms often involve a disproportionation reaction. For instance, via disproportionation, a methyl group from one molecule of toluene may be transferred to a second molecule of toluene to form one molecule of benzene and one molecule of xylene. An ongoing need exists for catalysts that maximize the production of xylene and benzene via the disproportionation of toluene.

Embodiments of the present disclosure meet this need by using hierarchical zeolite catalysts to convert toluene to benzene and xylenes. The method includes contacting toluene with a hierarchical zeolite catalyst in the presence of hydrogen gas to produce benzene and xylene via toluene disproportionation. The hierarchical zeolite catalyst includes mesoporous Mordenite, which contains a plurality of mesopores. At least a portion of the plurality of mesopores are arranged in an MCM-41 framework.

Any combinations of the various embodiments and implementations disclosed herein can be used in a further embodiment, consistent with the disclosure. Additional features and advantages of the described embodiments will be set forth in the detailed description which follows, and in part will be readily apparent to those skilled in the art from that description or recognized by practicing the described embodiments, including the detailed description which follows, the claims, as well as the appended drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 is an X-Ray Diffraction (“XRD”) pattern of a mesoporous Mordenite in accordance with one or more embodiments of the present disclosure. That at least a portion of the plurality of mesopores are arranged in an MCM-41 framework is evidenced by a low angle signal at 20=2-5.

FIG. 2 is a plot comparing, as a function of reaction time, the xylene yield from a toluene disproportionation reaction using a conventional Mordenite catalyst and the xylene yield from a toluene disproportionation reaction using an inventive hierarchical Mordenite catalyst, as described herein.

### DETAILED DESCRIPTION

Reference will now be made in detail to embodiments of a method for using a hierarchical zeolite catalyst for the disproportionation of toluene to produce benzene and xylenes. The hierarchical zeolite catalyst comprises mesoporous Mordenite, wherein the mesoporous Mordenite comprises at least some mesopores uniformly arranged in an MCM-41 framework. Without being bound by any theory, it is believed that the introduction of mesopores into the Mordenite structure increases the probability that larger molecules in a feed stream, such as toluene, will have better access to the active sites inside the mesopores of the hierarchical zeolite catalyst. Moreover, it is believed that the mesopores allow for more facile transportation of larger molecules in a product stream, such as xylenes, which reduces the time that these molecules spend inside the zeolite matrix and, accordingly, reduces the probability of coke formation and other undesirable reactions.

In embodiments, a stream comprising toluene is introduced into a reactor, which includes the hierarchical zeolite catalyst and, optionally, a hydrogen gas feed. The stream comprising toluene contacts the hierarchical zeolite catalyst to produce benzene and xylene via toluene disproportionation. Contacting the stream comprising toluene with a hierarchical zeolite catalyst in the presence of hydrogen gas produces a hydrocarbon effluent comprising benzene, xylenes, unreacted toluene, and other hydrocarbons. In embodiments, the benzene and xylenes are collected and separated out from the hydrocarbon effluent.

In embodiments of the method disclosed herein, the stream comprising toluene may comprise from 99 wt. % to 100 wt. % of toluene.

While the disproportionation of toluene to benzene and xylene results in no net change in hydrogen, a hydrogen co-feed or input may be employed in order to prolong the useful life of the catalyst. Accordingly, hydrogen gas may be supplied to the reactor in which the toluene disproportionation reaction is carried out. In embodiments disclosed herein, the molar ratio of hydrogen gas to toluene may be from about 1 to about 4.

In embodiments, the method disclosed herein comprises a weight hourly space velocity (“WHSV”) of the stream comprising toluene of from about 1.0 h⁻¹ to about 5.0 h⁻¹.

As would be familiar to the skilled person, various reactors—such as fluidized bed reactors, fixed bed reactors, and the like—are considered suitable for embodiments of the disclosed method. In embodiments, the method disclosed herein comprises a reactor pressure of from about 500 kPa to about 3000 kPa, such as from about 500 kPa to about 2000 kPa, from about 500 kPa to about 1000 kPa, from about 1000 kPa to about 3000 kPa, from about 1000 kPa to about 2000 kPa, or from about 2000 kPa to about 3000 kPa. In embodiments, the method disclosed herein comprises a reactor temperature of from about 200° C. to about 500° C., such as from about 200° C. to about 400° C., from about 200° C. to about 300° C., from about 300° C. to about 500° C., or from about 300° C. to about 400° C.

In embodiments, the hierarchical zeolite catalyst used in the method disclosed herein comprises different phases of porosity. First, according to embodiments, the hierarchical zeolite catalyst comprises a microporous structure, or “microphase,” comprised of micropores having a diameter less than or equal to about 2 nanometers (“nm”). The hierarchical zeolite catalyst used in the method disclosed herein comprises a Mordenite microphase. Mordenite is a zeolite mineral and is one of a number of catalysts commonly employed in the disproportionation of alkyl aromatic compounds, such as toluene. Mordenite has the chemical formula XAl₂Si₁₀O₂₄·7H₂O, in which X can be Ca, Na₂, or K₂. The molecular framework of Mordenite contains chains of five-membered rings of linked silicate and aluminate tetrahedra, and its crystalline structure presents mono-directional channels defined by twelve-membered rings. The micropore system of Mordenite consists of main channels of 6.5×7.0 Å, which are connected by tortuous pores of 2.6 5.7 Å. Mordenite zeolite can be synthesized starting from its components' precursors, with a broader silica-to-alumina molar ratio in the range of 6 to 30.

Second, in embodiments, the hierarchical zeolite catalyst used in the method disclosed herein further comprises a mesoporous structure, or “mesophase,” comprised of mesopores having a diameter of greater than about 2 nm and less than or equal to about 50 nm. As used herein, “mesoporous Mordenite” refers to a zeolite material comprising a Mordenite microporous phase and a mesophase, wherein the mesophase comprises a plurality of mesopores. In embodiments, the mesoporous Mordenite comprises an “ordered/disordered mesophase.” As used herein, an “ordered mesophase” is a crystalline zeolite uniform arrangement of mesopores and a “disordered mesophase” is a non-uniform arrangement of mesopores. As used herein, an “ordered/disordered mesophase” is a mesophase where at least some of the mesopores are uniformly arranged and at least some of the mesopores are not uniformly arranged.

In embodiments, the hierarchical zeolite catalyst used in the method disclosed herein comprises an ordered/disordered mesophase comprising a plurality of mesopores, wherein at least some of the plurality of mesopores are uniformly arranged. In embodiments, the at least some of the plurality of mesopores are arranged in a MCM-41 framework. As defined, MCM-41 (MCM=Mobil Composite of Matter) is an amorphous mesoporous silica with a hexagonal mesopore arrangement, consisting of a regular arrangement of cylindrical mesopores that form a one-dimensional pore system. In embodiments disclosed herein, the MCM-41 mean mesopore diameter may be in the range of from about 2 nm to about 50 nm.

In embodiments of the method disclosed herein, the MCM-41 arrangement of the at least some of the plurality mesopores of the mesoporous Mordenite may be indicated by one or more XRD peaks at 2θ=2-5.

In embodiments of the method disclosed herein, the molar ratio of silica-to-alumina in the mesoporous Mordenite may be from 18 to 500, such as from about 18 to about 100, from about 100 to about 200, from about 200 to about 300, from about 300 to about 400, or from about 400 to about 500.

In embodiments of the method disclosed herein, the mesoporous Mordenite may have a total surface area (“Sₜₒₜ”), a micropore surface area (“S_{mic}”), and an external surface area (“Sₑₓₜ”) defined by a Brunauer-Emmett-Teller (BET) analysis. In embodiments, the mesoporous Mordenite may have an Sₜₒₜ of from about 200 meters² per gram (“m²/g”) to about 1500 m²/g, such as from about 200 m²/g to about 500 m²/g, from about 500 m²/g to about 100 m²/g, or from about 1000 m²/g to about 1500 m²/g. In embodiments, the mesoporous Mordenite may have an S_{mic} of from about 100 m²/g to about 500 m²/g, such as from about 100 m²/g to about 200 m²/g, from about 200 m²/g to about 300 m²/g, from about 300 m²/g to about 400 m²/g, or from about 400 m²/g to about 500 m²/g. In embodiments, the mesoporous Mordenite may have an Sₑₓₜ of from about 200 m²/g to about 600 m²/g, such as from about 200 m²/g to about 300 m²/g, from about 300 m²/g to about 400 m²/g, from about 400 m²/g to about 500 m²/g, or from about 500 m²/g to about 600 m²/g.

In embodiments of the method disclosed herein, the mesoporous Mordenite may have a total pore volume (“Vₜₒₜ”), defined using BET analysis; a micropore volume (“V_{mic}”), defined using a 1-plot method; and a mesopore volume (“Vₘₑₛ”), that can be calculated from about the difference in Vₜₒₜ and V_{mic}. In one or more embodiments, the mesoporous Mordenite may have a Vₜₒₜ of from about 0.01 to about 5.0 cubic centimeters per gram (“cm³/g”), such as from about 0.01 cm³/g to about 1.0 cm³/g, from about 1.0 cm³/g to about 2.0 cm³/g, from about 2.0 cm³/g to about 3.0 cm³/g, from about 3.0 cm³/g to about 4.0 cm³/g, or from about 4.0 cm³/g to about 5.0 cm³/g. In embodiments, the mesoporous Mordenite may have a V_{mic} of from about 0.01 cm³/g to about 2.0 cm³/g, such as from about 0.01 cm³/g to about 0.5 cm³/g, from about 0.5 cm³/g to about 1.0 cm³/g, from about 1.0 cm³/g to about 1.5 cm³/g, or from about 1.5 cm³/g to about 2.0 cm³/g. In embodiments, the mesoporous Mordenite may have a Vₘₑₛ of from about 0.10 cm³/g to about 3.0 cm³/g, such as from about 0.10 cm³/g to about 0.50 cm³/g, from about 0.50 cm³/g to about 1.0 cm³/g, from about 1.0 cm³/g to about 1.5 cm³/g, from about 1.5 cm³/g to about 2.0 cm³/g, from about 2.0 cm³/g to about 2.5 cm³/g, or from about 2.5 cm³/g to about 3.0 cm³/g.

In embodiments of the method disclosed herein, the mesoporous Mordenite may have an average mesopore pore size defined using the Barrett-Joyner-Halenda (BJH) model applied to the adsorption branch of an N₂ isotherm. In embodiments, the average mesopore pore size of the mesoporous Mordenite may be from about 2 nanometers (“nm”) to about 50 nm.

In embodiments of the method disclosed herein, the hierarchical zeolite catalyst may be impregnated with one or more active metals for catalysis. Without being bound by any theory, it is believed that the presence of the active metal reduces unwanted side reactions and the formation of heavy molecules, both of which may occur when toluene disproportionation takes place in the presence of a zeolite without an active metal. In embodiments, the active metal may be molybdenum, platinum, rhenium, nickel, cobalt, or combinations thereof. In one or more embodiments, the active metal may be molybdenum. The active metal component may exist, in embodiments, within the final hierarchical zeolite catalyst as an elemental metal. The metal component may exist, in embodiments, within the final hierarchical zeolite catalyst as a compound, such as a metal oxide, a metal sulfide or a metal halide, in chemical combination with one or more of the other ingredients of the hierarchical zeolite catalyst, or as the active elemental metal. In embodiments, the metal oxide may be reduced under hydrogen pressure and an elevated temperature, such as 450° C., to form the active elemental metal. The active metal component may be present in the final hierarchical zeolite catalyst in any amount that is catalytically effective, generally from about 0.01 wt % to about 6.0 wt % or from about 2 wt % to about 5 wt % of the zeolite catalyst.

In embodiments of the method disclosed herein, the hierarchical zeolite catalyst may comprise one or more zeolites in addition to the mesoporous Mordenite. In embodiments, the hierarchical zeolite catalyst may comprise at least 50 wt. %, at least 60 wt. %, at least 70 wt. %, at least 80 wt. %, at least 90 wt. %, at least 95 wt. %, at least 99 wt. %, or even 100 wt. % of the mesoporous Mordenite, based on the total weight of zeolites in the hierarchical zeolite catalyst.

In embodiments, the hierarchical zeolite catalyst may comprise less than 20 wt. %, less than 15 wt. %, less than 10 wt. %, less than 5 wt. %, or less than 1 wt. % of ZSM-5. In embodiments, the hierarchical zeolite catalyst may not comprise ZSM-5. Without intending to be bound by any particular theory, it is believed that inclusion of the ZSM-5 in the hierarchical zeolite catalyst may reduce the production of benzene and xylene via toluene disproportionation compared to methods that include a hierarchical zeolite catalyst having an increased amount of mesoporous Mordenite and a decreased amount of ZSM-5.

In embodiments, the hierarchical zeolite catalyst may comprise the mesoporous Mordenite in an amount of at least 30 wt. %, at least 40 wt. %, at least 50 wt. %, at least 60 wt. %, at least 70 wt. %, at least 80 wt. %, or at least 90 wt. %, based on the total weight of the hierarchical zeolite catalyst. In embodiments, the hierarchical zeolite catalyst may comprise the mesoporous Mordenite in an amount from 30 wt. % to 40 wt. %, from 40 wt. % to 50 wt. %, from 50 wt. % to 60 wt. %, from 60 wt. % to 70 wt. %, from 70 wt. % to 80 wt. %, from 80 wt. % to 90 wt. %, from 90 wt. % to 100 wt. %, or any ranges and sub-ranges between any two of the foregoing values.

In embodiments, the hierarchical zeolite catalyst may comprise the mesoporous Mordenite in an amount of at least 30 wt. %, at least 40 wt. %, at least 50 wt. %, at least 60 wt. %, at least 70 wt. %, at least 80 wt. %, or at least 90 wt. %, based on the total weight of the hierarchical zeolite catalyst. In embodiments, the hierarchical zeolite catalyst may comprise the mesoporous Mordenite in an amount from 30 wt. % to 40 wt. %, from 40 wt. % to 50 wt. %, from 50 wt. % to 60 wt. %, from 60 wt. % to 70 wt. %, from 70 wt. % to 80 wt. %, from 80 wt. % to 90 wt. %, from 90 wt. % to 100 wt. %, or any ranges and sub-ranges between any two of the foregoing values, based om the total weight of the hierarchical zeolite catalyst.

In embodiments, the hierarchical zeolite catalyst may comprise an alumina binder in an amount from 0 wt. % to 10 wt. %, from 10 wt. % to 20 wt. %, from 20 wt. % to 30 wt. %, from 30 wt. % to 40 wt. %, from 40 wt. % to 50 wt. %, from 50 wt. % to 60 wt. %, from 60 wt. % to 70 wt. % or any ranges and sub-ranges between any two of the foregoing values, based om the total weight of the hierarchical zeolite catalyst.

In embodiments, the hierarchical zeolite catalyst may comprise, consist essentially of, or consist of from 50 wt. % 80 wt. % of the mesoporous Mordenite, from 20 wt. % to 45 wt. % of an alumina binder, from 2 wt. % to 6 wt. % of the active metal. In embodiments, the hierarchical zeolite catalyst may comprise, consist essentially of, or consist of from 50 wt. % 80 wt. % of the mesoporous Mordenite, from 20 wt. % to 45 wt. % of an alumina binder, from 2 wt. % to 6 wt. % of the active metal, and less than 5 wt. % of ZSM-5. In embodiments, the hierarchical zeolite catalyst may comprise, consist essentially of, or consist of from 50 wt. % 80 wt. % of the mesoporous Mordenite, from 20 wt. % to 45 wt. % of an alumina binder, from 2 wt. % to 6 wt. % of the active metal, and less than 1 wt. % of ZSM-5. In embodiments, the hierarchical zeolite catalyst may comprise, consist essentially of, or consist of from 50 wt. % 80 wt. % of the mesoporous Mordenite, from 20 wt. % to 45 wt. % of an alumina binder, from 2 wt. % to 6 wt. % of the active metal, and may not comprise ZSM-5.

In embodiments of the method disclosed herein, the hierarchical zeolite catalyst comprising mesoporous Mordenite may be produced via a base-mediated degrading of a “parent” Mordenite into multiple oligomeric units, followed by a surfactant-mediated re-assembly of the oligomeric units. The degrading and re-assembly steps are controlled to minimize or avoid the amorphization of the parent Mordenite. The resulting mesoporous Mordenite comprises a plurality of mesopores, wherein at least some of the plurality of mesopores are arranged in an MCM-41 framework.

In embodiments of the method disclosed herein, the hierarchical zeolite catalyst comprising mesoporous Mordenite may be produced by first dissolving preformed Mordenite in an alkaline solution, while heating, stirring, or both, to yield an alkaline Mordenite solution. In embodiments, prior to the addition of the Mordenite, the alkaline solution comprises from about 0.1 M to about 0.6 M sodium hydroxide. In embodiments, the dissolving the preformed Mordenite in an alkaline solution may result in the preformed Mordenite being degraded into multiple oligomeric units.

While heating, stirring, or both, a surfactant is added to the alkaline zeolite solution to form a Mordenite-surfactant mixture. In embodiments, the Mordenite-surfactant mixture comprises from about 1 wt % to about 8 wt % of cetyltrimethyl ammonium bromide. The Mordenite-surfactant mixture is cooled and the pH is adjusted to about 9.0, and then the Mordenite-surfactant mixture is hydrothermally treated for a duration of time. In embodiments, hydrothermally treating the Mordenite-surfactant mixture for a duration of time comprises heating and stirring the Mordenite-surfactant mixture at an elevated temperature. In embodiments, the elevated temperature may be about 100° C., from about 50° C. to about 200° C., or from about 80° C. to about 120° C. for the duration of time. In embodiments, the duration of time may be from about 30 minutes to about 48 hours. In embodiments, during hydrothermal treatment, the multiple oligomeric units of the Mordenite may reassemble around the micelles of the surfactant, forming mesopores with surfactant trapped inside.

A solid mesoporous Mordenite product comprising trapped surfactant in the mesopores is separated from the Mordenite-surfactant mixture. Finally, the solid mesoporous Mordenite product is dried and calcined. Calcining removes the trapped surfactant from the solid mesoporous Mordenite product and yields a mesoporous Mordenite wherein the mesoporous Mordenite comprises a plurality of mesopores and at least some of the plurality of mesopores are uniformly arranged in an MCM-41 framework.

The mesoporous Mordenite may, in embodiments, be subjected to ion-exchange and extruded by mixing the mesoporous Mordenite with an alumina binder. As disclosed herein, the mesoporous Mordenite may be impregnated with an active metal.

### EXAMPLES

The various embodiments of the method described will be further clarified by the following examples. The examples are illustrative in nature, and should not be understood to limit the subject matter of the present disclosure.

### Example 1: Synthesis of Catalyst a (Hierarchical Catalyst Comprising Mesoporous Mordenite and MCM-41 Framework)

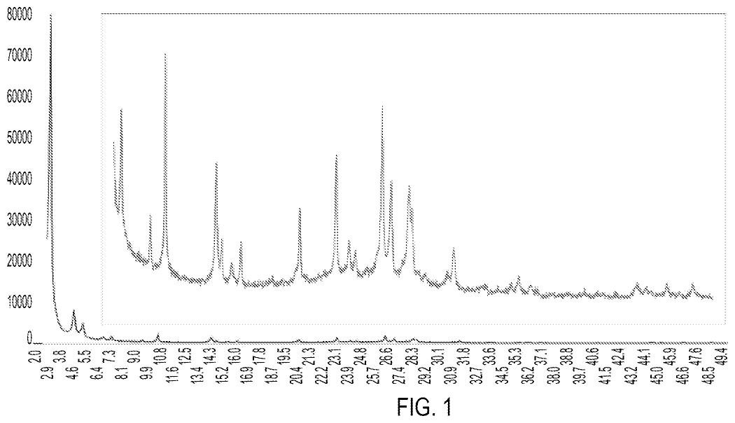

Catalyst A was prepared as follows: In a glass reactor, 3 grams (“g”) of Mordenite (Si/Al ratio 30, HSZ-660 HOA) was dissolved in 0.40 moles per liter (“M”) of an aqueous NaOH solution with gradual heating to 100 degrees Celsius (“° C.”) and stirring for 24 hours (“h”). The heating was carried out in the presence of cetyltrimethylammonium bromide (“CTAB”) surfactant (4.45 wt %) to form a mesoporous Mordenite comprising a plurality of mesopores in an MCM-41 framework. The mixture was cooled down and the pH was adjusted to 9.0 through the addition of dilute sulfuric acid (2 N). The mixture was stirred for 24 h at room temperature, and then held at 100° C. for 24 h. The solid mesoporous Mordenite product was filtered, washed thoroughly using distilled water, dried at 80° C. overnight, and then calcined at 550° C. for 6 h to remove the CTAB from the mesopores. The obtained mesoporous Mordenite was ion-exchanged thrice with 0.05 M NH₄NO₃ solution at 80° C. for 5 h. The resulting mesoporous Mordenite was made into extrudates by mixing 67 wt % mesoporous Mordenite and 33 wt % alumina binder (Cataloid AP-3, obtained from CCIC, Japan). The extruded mesoporous Mordenite was loaded with 4 wt % of molybdenum in the form of ammonium molybdate tetrahydrate through a wet impregnation technique. The impregnated mesoporous Mordenite was calcined at 550° C. for 5 h. The resulting catalyst of Example 1 is denoted as Catalyst A. The XRD pattern of Catalyst A is shown in FIG. 1. The low angle signal at 2θ=2-4.5 indicates the presence of the MCM-41 mesoporous structure.

### Example 2: Preparation of Catalyst B (Mordenite) and Synthesis of Catalyst C (Hierarchical Catalyst Comprising Mesoporous Mordenite and ZSM-5)

Catalyst B was prepared by impregnating a commercially available Mordenite (HSZ660 HOA) with 4 wt % molybdenum.

Catalyst C was prepared as follows: In a glass reactor, 3 grams (“g”) of ZSM-5 (Si/Al ratio 30, HSZ-840 HOA) was dissolved in 0.40 moles per liter (“M”) of an aqueous NaOH solution with gradual heating to 100 degrees Celsius (“° C.”) and stirring for 24 hours (“h”). The heating was carried out in the presence of cetyltrimethylammonium bromide (“CTAB”) surfactant (4.45 wt %) to form a mesoporous ZSM-5 comprising a plurality of mesopores. The mixture was cooled down and the pH was adjusted to 9.0 through the addition of dilute sulfuric acid (2 N). The mixture was stirred for 24 h at room temperature, and then held at 100° C. for 24 h. The solid mesoporous ZSM-5 product was filtered, washed thoroughly using distilled water, dried at 80° C. overnight, and then calcined at 550° C. for 6 h to remove the CTAB from the mesopores. The obtained mesoporous ZSM-5 was ion-exchanged thrice with 0.05 M NH₄NO₃ solution at 80° C. for 5 h. The mesoporous Mordenite formed in Example A (prior to the forming of extrudates) was mixed with the resulting mesoporous ZSM-5 at a weight ratio of 3:1 (Mordenite: ZSM-5). The mixture Mordenite and ZSM-5 was made into extrudates by mixing 67 wt % mesoporous Mordenite/ZSM-5 mixture and 33 wt % alumina binder (Cataloid AP-3, obtained from CCIC, Japan). The extruded mesoporous Mordenite/ZSM-5 was loaded with 4 wt % of molybdenum in the form of ammonium molybdate tetrahydrate through a wet impregnation technique. The impregnated mesoporous Mordenite/ZSM-5 was calcined at 550° C. for 5 h. The resulting catalyst is denoted as Catalyst C.

### Example 3: Conversion of Toluene to Benzene and Xylene

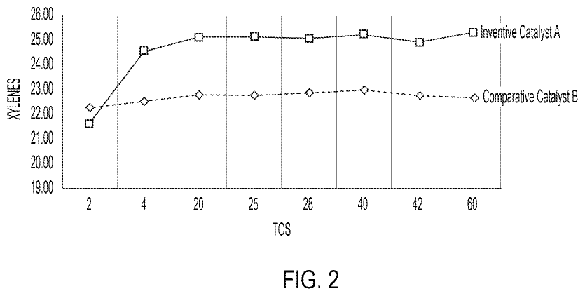

The catalytic activity of Catalyst A was compared to that of Catalyst B and Catalyst C. The structural parameters of Catalyst A, Catalyst B, and Catalyst C are provided in Table 1. The catalytic activity studies were performed in bench top vertical reactors using commercial toluene as feed. To determine catalytic activity, each reactor was loaded with 1.0 g of a catalyst (Catalyst A, B, or C) in the isothermal zone of reactor and with inert silicon carbide in the lower and upper parts of the reactor. The total volume of each reactor was 20 milliliters (“mL”). The catalyst was activated and reduced under a 50 mL/min flow of pure hydrogen gas at 450° C. and was kept at this temperature for 2 hours. Then, the pressure of the reactor was increased to 25 bar (2500 kPa) and the flow of feedstock was started at 4.2 g/h. The reaction was allowed to run for at least one hour at this temperature before collecting a product sample and was run for at least 50 additional hours without loss of activity before termination. The data for each reactor is shown in Table 2. FIG. 2 also shows the production of xylene as a function of reaction time using Catalyst A, Catalyst B, and Catalyst C corresponding to trace 210, 220, and 230, respectively.

TABLE 1

| | | | Sₜₒₜ | S_{mic} | Sₑₓₜ | Vₜₒₜ | V_{mic} | Vₘₑₛ |
|---|---|---|---|---|---|---|---|---|
| Catalyst | Composition | Si/Al | (m²g⁻¹) | (m²g⁻¹) | (m²g⁻¹) | (cm³g⁻¹) | (cm³g⁻¹) | (cm³g⁻¹) |
| Catalyst | Mesoporous | 27 | 622 | 240 | 382 | 0.7724 | 0.1390 | 0.6330 |
| A | Mordenite/MCM-41 | | | | | | | |
| | w/4 wt % Mo | | | | | | | |
| Catalyst | Commercial Mordenite | 30 | 402 | 349 | 53 | 0.5519 | 0.1800 | 0.1010 |
| B | w/4 wt % Mo | | | | | | | |
| Catalyst | Mesoporous | Mordenite: | 641 | 220 | 421 | 0.8573 | 0.099 | 0.7583 |
| C | Mordenite/Mesoporous | 27 | | | | | | |
| | ZSM-5 (3:1) w/4 wt % Mo | ZSM-5: 30 | | | | | | |

TABLE 2

| | Catalyst A | | | | | | | |
|---|---|---|---|---|---|---|---|---|
| | (Mesoporous Mordenite/MCM-41 w/4 wt % Mo) | | | | | | | |
| Time (h) | 2 | 4 | 20 | 25 | 28 | 40 | 42 | 60 |
| Temp (° C.) | 400 | 400 | 400 | 400 | 400 | 400 | 400 | 400 |
| Conversion (%) | 47.33 | 54.77 | 54.82 | 55.65 | 55.31 | 55.12 | 55.82 | 55.58 |
| Xylenes (%) | 21.63 | 24.62 | 25.16 | 25.15 | 25.10 | 25.27 | 24.89 | 25.35 |
| Benzene (%) | 20.87 | 24.00 | 24.03 | 24.41 | 23.85 | 23.86 | 24.81 | 24.80 |
| Ethyl Benzene (%) | 0.42 | 0.55 | 0.43 | 0.55 | 0.55 | 0.56 | 0.57 | 0.57 |
| C₉ Hydrocarbons (%) | 2.97 | 3.88 | 4.22 | 4.08 | 4.01 | 4.15 | 4.07 | 4.07 |
| Light Hydrocarbons (%) | 1.00 | 1.10 | 1.35 | 0.83 | 0.77 | 0.63 | 0.76 | 0.78 |
| Unreacted Toluene (%) | 52.67 | 45.23 | 44.22 | 44.35 | 44.69 | 44.88 | 44.18 | 44.42 |
| | Catalyst B | | | | | | | |
| | (Commercial Mordenite w/4 wt % Mo) | | | | | | | |
| Time (h) | 2 | 4 | 20 | 25 | 28 | 40 | 42 | 60 |
| Temp (° C.) | 400 | 400 | 400 | 400 | 400 | 400 | 400 | 400 |
| Conversion (%) | 52.03 | 52.27 | 52.34 | 52.42 | 52.03 | 52.27 | 52.34 | 52.42 |
| Xylenes (%) | 22.27 | 22.56 | 22.80 | 22.80 | 22.89 | 23.00 | 22.77 | 22.65 |
| Benzene (%) | 22.01 | 21.52 | 22.00 | 22.54 | 22.37 | 22.53 | 22.45 | 22.44 |
| Ethyl Benzene (%) | 0.52 | 0.51 | 0.50 | 0.48 | 0.47 | 0.52 | 0.51 | 0.50 |
| C₉ Hydrocarbons (%) | 5.05 | 5.16 | 5.00 | 4.85 | 5.05 | 5.16 | 5.00 | 5.17 |
| Light Hydrocarbons (%) | 1.65 | 1.63 | 1.27 | 1.49 | 1.42 | 1.51 | 1.34 | 1.54 |
| Unreacted Toluene (%) | 47.97 | 47.73 | 47.66 | 47.58 | 47.97 | 47.73 | 47.66 | 47.58 |

| | Catalyst C | | | | |
|---|---|---|---|---|---|
| | (Mesoporous Mordenite/Mesoporous ZSM-5 (3:1) w/4 wt % Mo) | | | | |
| Time (h) | 2 | 4 | 18 | 21 | 31 |
| Temp (° C.) | 400 | 400 | 400 | 400 | 400 |
| Conversion (%) | 35.60 | 38.40 | 43.85 | 44.10 | 43.9 |
| Xylenes (%) | 15.20 | 15.80 | 18.45 | 18.60 | 18.55 |
| Benzene (%) | 13.10 | 13.30 | 15.23 | 15.50 | 15.62 |
| Ethyl Benzene (%) | 0.10 | 0.10 | 0.85 | 0.90 | 0.88 |
| C₉ Hydrocarbons (%) | 1.80 | 1.92 | 2.50 | 2.85 | 2.55 |
| Light Hydrocarbons (%) | 0.25 | 0.20 | 0.38 | 0.42 | 0.41 |
| Unreacted Toluene (%) | 68.85 | 69.48 | 62.59 | 61.32 | 61.70 |

The data in Table 1 reveal that toluene conversion using Catalyst A resulted in a higher yield of benzene and xylene than did toluene conversion using Catalyst B or Catalyst C. For example, after 60 hours, a sample from the reaction using Catalyst A comprised 25.35% xylene, while a sample from the reaction using Catalyst B comprised 22.65% xylene. After 20 hours, a sample from the reaction using Catalyst A comprised 25.16% xylene, while after 21 hours a sample from the reaction using Catalyst C comprised 18.60% xylene. These differences can be appreciated by the data plot shown in FIG. 2. Likewise, the percentage of benzene in a sample from the reaction using Catalyst A (24.80%) was nearly 2.5% higher than the percentage of benzene in a sample from the reaction using Catalyst B (22.44%) after 60 hours; the percentage of benzene in a sample from the reaction using Catalyst A (24.03%) was nearly 9% higher than the percentage of benzene in a sample from the reaction using Catalyst C (15.50%) after 20 hours and 21 hours, respectively.

Moreover, the product sample from the reaction using Catalyst A comprised a lower percentage of other hydrocarbon by-products, including heavier hydrocarbons, and a lower percentage of unreacted toluene than did the product sample from the analogous reactions using Catalyst B or Catalyst C. The differences in product outcomes are believed to result from the increased surface area of Catalyst A and the accessibility of active sites within the mesopores.

The present disclosure includes one or more non-limiting aspects.

A first aspect includes a method of converting toluene to benzene and xylene, the method including contacting a stream comprising toluene with a hierarchical zeolite catalyst in the presence of hydrogen gas in a reactor to produce the benzene and xylene via toluene disproportionation, wherein the hierarchical zeolite catalyst includes mesoporous Mordenite, wherein the mesoporous Mordenite includes a plurality of mesopores and at least some of the plurality of mesopores are uniformly arranged in an MCM-41 framework.

A second aspect includes the method according to the first aspect, wherein the reactor comprises a reactor pressure of from about 500 kPa to about 3000 kPa.

A third aspect includes the method according to the first aspect or the second aspect, wherein the reactor comprises a reactor temperature of from about 200° C. to about 500° C.

A fourth aspect includes the method according to any one of the first through third aspects, wherein a weight hourly space velocity of the stream comprising toluene of from about 1.0 h⁻¹ to about 5.0 h⁻¹.

A fifth aspect includes the method according to any one of the first through fourth aspects, wherein a molar ratio of hydrogen gas to toluene is from about 1 to about 4.

A sixth aspect includes the method according to any one of the first through fifth aspects, wherein the at least a portion of the mesopores uniformly arranged in an MCM-41 framework produce one or more X-ray diffraction peaks at 20=2-5.

A seventh aspect includes the method according to any one of the first through sixth aspects, wherein the mesoporous Mordenite includes a silica-to-alumina molar ratio of from about 18 to about 100.

An eighth aspect includes the method according to any one of the first through seventh aspects, wherein the mesoporous Mordenite includes a total surface area of from about 400 m²/g to about 800 m²/g, a micropore surface area of from about 150 m²/g to about 350 m²/g, and an external surface area of from about 300 m²/g to about 500 m²/g.

A ninth aspect includes the method according to any one of the first through eighth aspects, wherein the mesoporous Mordenite includes a total pore volume of from about 0.10 cm³/g to about 3.0 cm³/g, a micropore volume of from about 0.01 cm³/g to about 0.30 cm³/g, and a mesopore volume of from about 0.10 cm³/g to about 2.0 cm³/g.

A tenth aspect includes the method according to any one of the first through ninth aspects, wherein the hierarchical zeolite catalyst further includes one or more active metals comprising platinum, rhenium, nickel, cobalt, or combinations thereof.

An eleventh aspect includes the method according to the tenth aspect, wherein the hierarchical zeolite catalyst is impregnated with the one or more active metals via a wet impregnation technique.

A twelfth aspect includes the method according to the tenth aspect, wherein the hierarchical zeolite catalyst includes from about 0.01 wt % to about 6.0 wt % of the one or more active metals.

A thirteenth aspect includes the method according to any one of the first through twelfth aspects, wherein the hierarchical zeolite catalyst further includes molybdenum.

A fourteenth aspect includes the method according to any one of the first through thirteenth aspects, wherein the contacting the stream including toluene with a hierarchical zeolite catalyst in the presence of hydrogen gas produces a hydrocarbon effluent comprising benzene, xylenes, unreacted toluene, and other hydrocarbons and the method further includes collecting benzene and xylenes from the hydrocarbon effluent.

A fifteenth aspect includes the method according to any one of the first through fourteenth aspects, wherein the hierarchical zeolite catalyst is produced bydissolving in an alkaline solution, while heating, stirring, or both, a Mordenite to yield an alkaline Mordenite solution; adding to the alkaline Mordenite solution, while heating, stirring, or both, a surfactant to form a Mordenite-surfactant mixture; cooling and adjusting the pH of the Mordenite-surfactant mixture; hydrothermally treating the Mordenite-surfactant mixture for a duration of time; separating from the Mordenite-surfactant mixture a solid mesoporous Mordenite product, wherein the solid mesoporous Mordenite product comprises surfactant; and drying and calcining the solid mesoporous Mordenite product to produce the hierarchical zeolite catalyst including the mesoporous Mordenite, wherein the mesoporous Mordenite includes a plurality of mesopores and at least some of the plurality of mesopores are uniformly arranged in an MCM-41 framework.

A sixteenth aspect includes the method according to the fifteenth aspect, further including subjecting the hierarchical zeolite catalyst including the mesoporous Mordenite to ion-exchange.

A seventeenth aspect includes the method according to the sixteenth aspect, further including extruding the hierarchical zeolite catalyst including the mesoporous Mordenite by mixing it with an alumina binder.

An eighteenth aspect includes the method according to the fifteenth aspect, wherein the alkaline solution includes from about 0.1 M to about 0.6 M sodium hydroxide prior to the addition of the Mordenite.

A nineteenth aspect includes the method according to the fifteenth aspect, wherein the Mordenite-surfactant mixture includes from about 1 wt % to about 8 wt % of cetyltrimethyl ammonium bromide.

A twentieth aspect includes the method according to the fifteenth aspect, wherein hydrothermally treating the Mordenite-surfactant mixture for a duration of time includes heating and stirring the Mordenite-surfactant mixture at about 100° C. for the duration of from 30 minutes to 48 hours.

Having described the subject matter of the present disclosure in detail and by reference to specific embodiments, it is noted that the various details disclosed in the present disclosure should not be taken to imply that these details relate to elements that are essential components of the various embodiments described in the present disclosure. Further, it will be apparent that modifications and variations of the particular embodiments disclosed, or to the best mode contemplated for carrying out this invention, are possible without departing from the scope of the present disclosure, including, but not limited to, all embodiments falling within the scope of the appended claims. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. For example, a chemical composition “consisting essentially” of a particular chemical constituent or group of chemical constituents should be understood to mean that the composition includes at least about 99.5% of a that particular chemical constituent or group of chemical constituents.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

1. A method of converting toluene to benzene and xylene, the method comprising contacting a stream comprising toluene with a hierarchical zeolite catalyst in the presence of hydrogen gas in a reactor to produce the benzene and xylene via toluene disproportionation, wherein the hierarchical zeolite catalyst comprises mesoporous Mordenite, wherein the mesoporous Mordenite comprises a plurality of mesopores and at least a portion of the plurality of mesopores are uniformly arranged in an MCM-41 framework.

2. The method of claim 1, wherein the reactor comprises a reactor pressure of from about 500 kPa to about 3000 kPa.

3. The method of claim 1, wherein the reactor comprises a reactor temperature of from about 200° C. to about 500° C.

4. The method of claim 1, wherein a weight hourly space velocity of the stream comprising toluene is from about 1.0 h⁻¹ to about 5.0 h⁻¹.

5. The method of claim 1, wherein a molar ratio of hydrogen gas to toluene is from about 1 to about 4.

6. The method of claim 1, wherein the at least a portion of the mesopores uniformly arranged in an MCM-41 framework produce one or more X-ray diffraction peaks at 2θ=2-5.

7. The method of claim 1, wherein the mesoporous Mordenite comprises a silica-to-alumina molar ratio of from about 18 to about 100.

8. The method of claim 1, wherein the mesoporous Mordenite comprises a total surface area of from about 400 m²/g to about 800 m²/g, a micropore surface area of from about 150 m²/g to about 350 m²/g, and an external surface area of from about 300 m²/g to about 500 m²/g.

9. The method of claim 1, wherein the mesoporous Mordenite comprises a total pore volume of from about 0.10 cm³/g to about 3.0 cm³/g, a micropore volume of from about 0.01 cm³/g to about 0.30 cm³/g, and a mesopore volume of from about 0.10 cm³/g to about 2.0 cm³/g.

10. The method of claim 1, wherein the hierarchical zeolite catalyst further comprises one or more active metals comprising molybdenum, platinum, rhenium, nickel, cobalt, or combinations therefrom.

11. The method of claim 10, wherein the hierarchical zeolite catalyst is impregnated with the one or more active metals via a wet impregnation technique.

12. The method of claim 10, wherein the hierarchical zeolite catalyst comprises from about 0.01 wt % to about 6.0 wt % of the one or more active metals.

13. The method of claim 1, wherein the hierarchical zeolite catalyst further comprises molybdenum.

14. The method of claim 1, wherein the contacting the stream comprising toluene with a hierarchical zeolite catalyst in the presence of hydrogen gas produces a hydrocarbon effluent comprising benzene, xylenes, unreacted toluene, and other hydrocarbons and the method further comprises collecting benzene and xylenes from the hydrocarbon effluent.

15. The method of claim 1, wherein the hierarchical zeolite catalyst is produced according to the following method:
dissolving in an alkaline solution, while heating, stirring, or both, a Mordenite to yield an alkaline Mordenite solution;
adding to the alkaline Mordenite solution, while heating, stirring, or both, a surfactant to form a Mordenite-surfactant mixture;
cooling and adjusting the pH of the Mordenite-surfactant mixture;
hydrothermally treating the Mordenite-surfactant mixture for a duration of time;
separating from the Mordenite-surfactant mixture a solid mesoporous Mordenite product, wherein the solid mesoporous Mordenite product comprises surfactant; and
drying and calcining the solid mesoporous Mordenite product to produce the hierarchical zeolite catalyst comprising the mesoporous Mordenite, wherein the mesoporous Mordenite comprises a plurality of mesopores and at least a portion of the plurality of mesopores are uniformly arranged in an MCM-41 framework.

16. The method of claim 15, further comprising subjecting the hierarchical zeolite catalyst comprising the mesoporous Mordenite to ion-exchange.

17. The method of claim 16, further comprising extruding the hierarchical zeolite catalyst comprising the mesoporous Mordenite by mixing it with an alumina binder.

18. The method of claim 15, wherein the alkaline solution comprises from about 0.1 M to about 0.6 M sodium hydroxide prior to the addition of the Mordenite.

19. The method of claim 15, wherein the Mordenite-surfactant mixture comprises from about 1 wt % to about 8 wt % of cetyltrimethyl ammonium bromide.

20. The method of claim 15, wherein hydrothermally treating the Mordenite-surfactant mixture for a duration of time comprises heating and stirring the Mordenite-surfactant mixture at about 100° C. for the duration of from 30 minutes to 48 hours.
